(19) United States
(12) Patent Application Publication
(10) Pub. No.: US 2022/0105222 A1
(43) Pub. Date: Apr. 7, 2022

(54) FAN DRIVEN AIR FRESHENER WITH FRAGRANCE END OF LIFE INDICATION

(71) Applicant: HENKEL IP & HOLDING GmbH, Dusseldorf (DE)

(72) Inventors: Terannie Vazquez Alvarez, Orange, CT (US); Mark A. Granja, Danbury, CT (US); Glenn William Kaye, Norwalk, CT (US); Edward Gilchrest, Oxford, CT (US)

(73) Assignee: HENKEL IP & HOLDING GMBH, Duesseldorf (DE)

(21) Appl. No.: 17/491,324

(22) Filed: Sep. 30, 2021

Related U.S. Application Data
(60) Provisional application No. 63/198,229, filed on Oct. 5, 2020.

(51) Int. Cl. A61L 9/12

(52) U.S. Cl. CPC A61L 9/122; A61L 2209/111; A61L 2209/134; A61L 2209/133

(57) ABSTRACT

A volatile substance distribution system includes a base unit and a removable capsule. The distribution system further includes an indicator system configured to indicate a usage condition of the capsule. The indicator system includes a capsule sensor, a timing device, and an output device. The capsule sensor is configured to detect that the capsule is in the engaged position. The timing device is configured to track a cumulative usage time of the capsule according to an amount of time the fan is in the operating condition with the capsule in the engaged position as detected by the capsule sensor. The output device is configured to provide an indication of the usage condition when the cumulative usage time reaches a predetermined threshold time.

### RELATED APPLICATION

The following claims priority to U.S. Provisional Patent Application 63/198229, filed Oct. 5, 2020, the entire disclosure of which is incorporated by reference.

### TECHNICAL FIELD

The following relates to a volatile substance distribution system and, more particularly, relates to an end-of-life indicator for a volatile substance distribution system, which indicates when the supply of volatile material in the system is in need of replenishment.

### BACKGROUND

There are various devices used to distribute volatile materials (e.g., perfumes, essential oils, insect repellant, etc.) into the air. Many devices include a unit that supports the volatile material for distribution into the air. Once the volatile material has been used up (vaporized), the unit may be replaced with a fresh supply of the volatile material.

However, there are various drawbacks with conventional systems. For example, a consumer may have a difficult time perceiving whether the volatile material has been used up. In the case of a fragrance, the consumer may need to rely on their sense of smell to determine whether the unit needs replacement; however, it may take a considerable amount of time before the consumer perceives the lack of aroma. Furthermore, persons are known to become “nose-blind” such that their ability to recognize a smell diminishes the longer a person has been smelling that scent. This “nose-blindness” can negatively affect the consumer's perception, thereby delaying replacement of the volatile material dispenser unit. Other volatile materials may be unscented, and the consumer may have no indication that replacement is necessary. These and other concerns may considerably limit the usefulness of these conventional systems.

Therefore, there exists a need for a volatile material distribution system that indicates end-of-life of the volatile material unit in an accurate and useful manner. Other desirable features and characteristics of the devices and methods of the present disclosure will become apparent from the subsequent detailed description and the appended claims, taken in conjunction with the accompanying drawings and the preceding background.

### BRIEF SUMMARY

Embodiments of a volatile substance distribution system are provided. In various embodiments, the system includes a housing configured to removably receive a capsule such that the capsule is in an engaged position with the housing. The capsule contains a volatile substance member. The distribution system includes a fan that is supported by the housing and that is selectively variable between a nonoperating condition and an operating condition. The fan, in the operating condition and with the capsule in the engaged position, drives air through the housing and the capsule. The distribution system further includes an indicator system configured to indicate a usage condition of the capsule. The indicator system includes a capsule sensor, a timing device, and an output device. The capsule sensor is configured to detect that the capsule is in the engaged position. The timing device is configured to track a cumulative usage time of the capsule according to an amount of time the fan is in the operating condition with the capsule in the engaged position as detected by the capsule sensor. The output device is configured to provide an indication of the usage condition when the cumulative usage time reaches a predetermined threshold time.

Embodiments of a method of operating a volatile substance distribution system are also provided. In some embodiments, the method includes engaging a capsule and a base unit in an engaged position. The capsule contains a volatile substance member and is removably engageable with the base unit. The base unit includes a housing that supports a fan. The fan is selectively variable between a nonoperating condition and an operating condition. The fan, in the operating condition and with the capsule in the engaged position, drives air through the housing and the capsule. The method also includes indicating, with an indicator system of the base unit, a usage condition of the capsule. This includes detecting, with a capsule sensor, that the capsule is in the engaged position. The method also includes tracking, with a timing device, a cumulative usage time of the capsule according to an amount of time the fan is in the operating condition with the capsule in the engaged position as detected by the capsule sensor. Furthermore, the method includes indicating, with an output device, the usage condition when the cumulative usage time reaches a predetermined threshold time.

The foregoing statements are provided by way of non-limiting example only. Various additional examples, aspects, and other features of embodiments of the present disclosure are encompassed by the present disclosure and described in more detail below.

### BRIEF DESCRIPTION OF THE DRAWINGS

At least one example of the present disclosure will hereinafter be described in conjunction with the following figures, wherein like numerals denote like elements, and:

FIG. 1 is a perspective view of a volatile substance distribution system according to example embodiments of the present disclosure;

FIG. 2 is a perspective view of a base unit of the system of FIG. 1;

FIG. 3 is a perspective view of a capsule of the system of FIG. 1;

FIG. 4 is an isometric section view of the base unit and the capsule of the system of FIG. 1;

FIG. 5 is a schematic diagram illustrating a method of operating an end-of-life indicator system of the volatile substance distribution system according to example embodiments; and

FIG. 6 is a flowchart illustrating the method of operating the end-of-life indicator system according to example embodiments.

For simplicity and clarity of illustration, descriptions and details of well-known features and techniques may be omitted to avoid unnecessarily obscuring the exemplary and non-limiting embodiments of the present disclosure described in the subsequent Detailed Description. It should further be understood that features or elements appearing in the accompanying figures are not necessarily drawn to scale unless otherwise stated.

### DETAILED DESCRIPTION

The following Detailed Description is merely exemplary in nature and is not intended to limit the present disclosure or the application and uses of the same. The term “exemplary,” as appearing throughout this document, is synonymous with the term “example” and is utilized repeatedly below to emphasize that the following description provides only multiple non-limiting examples of the present disclosure and should not be construed to restrict the scope of the present disclosure, as set-out in the Claims, in any respect.

Systems for distributing a volatile substance are provided, as are methods for operating and manufacturing such systems. Generally, the systems described herein may include a base unit and a capsule that may be removably engaged with the base unit. The capsule may contain a volatile substance member and may receive an airflow that is driven by a fan of the base unit. As the airflow moves through the capsule, the volatile substance may enter the airstream for distribution outside the system.

The system may include a usage timing device that records, tracks, etc. the amount of usage of the replaceable capsule. In other words, the device may record the amount of time that the capsule has been used (e.g., the cumulative amount of time that air has been driven through the capsule by the fan from the time the capsule was initially installed in the base unit). In some embodiments, the usage timing device may measure how long the fan is ON (i.e., rotating, operational, etc.) such that capsule usage is measured according to the amount of time that the fan is operational (i.e., driving air through the capsule). As such, the system can accurately measure capsule usage using simple, efficient, and effective computerized logic and processing.

A sensor device may be included that is operatively attached to the usage timing device. The sensor device may be configured to detect that the capsule is engaged with the base unit (e.g., removably supported on the base unit). The sensor device may be included on the base unit and may be actuated, activated, triggered, or otherwise switched by interaction with the capsule. For example, the sensor device may be a mechanical switch, and the weight of the capsule may be sufficient for actuating the switch, thereby indicating the presence of the capsule on the base unit. The switch may be maintained substantially in a single position (e.g., an actuated position) while the capsule remains seated in the base unit to register the capsule is available for use. As will be discussed these and other features of the sensor device (e.g., the switch) can be useful and effective. These features can improve recognition, detection, and/or sensing of the capsule. These features can also provide ergonomic benefits for the device.

The usage timing device and the sensor device may be operatively attached and/or incorporated in an end-of-life indicator system of the present disclosure. The “end-of-life” of the capsule may be interpreted as the end of useful life of the capsule and may represent a point at which the volatile substance is depleted from the capsule. The end-of-life indicator system operates according to the usage time that is tracked by the sensor device. Based on the tracked usage, the system may provide a warning indication that signals the end-of-life of the capsule is approaching (i.e., when the volatile material has been almost depleted). Also, based on the tracked usage, the system may provide an expiration indication that signals the end-of-life of the capsule has been reached (i.e., when the volatile material has been depleted). The indicator system may include one or more output devices, such as a visual output device, for providing these signals. The system may rely on a predetermined approximation, model, etc. of the ordinary useful life of the capsule for determining whether to provide the warning and/or the expiration indication.

The device may have user settings and controls for varying the operations of the system. For example, the fan may be selectively started by the user and then may turn OFF automatically after a set time interval. The device may offer the user two or more settings for changing this time interval. The device may also offer the user a manual shutoff option for forcing the fan to shut down and cut off power thereto. The usage tracking system of the present disclosure may account for these and/or other operations so that the end-of-life indicator system accurately and efficiently signals when the volatile materials need replacement.

Assuming that the capsule retains the switch in the fixed position, a timing device may track usage for the duration that the fan is powered ON. The timing device may stop timing when the fan is powered OFF. This measured amount of usage time may be stored in a computerized memory device. The timing device may measure more usage time (i.e., time periods when the fan powered ON), and this additional time may be added to the amount already stored in memory so as to calculate a cumulative amount of usage time. The updated cumulative usage time may be stored in memory. The end-of-life indicator may provide a signal (e.g., a visual and/or audio signal) based on the cumulative usage time that is tracked. The signal may be provided according to a predetermined usage model (e.g., fan ON for X hours=volatiles nearly depleted and fan ON for X+n hours=volatiles depleted). In some embodiments, a first signal (a warning signal) may be provided when the tracked cumulative usage time reaches a first predetermined level (X) based on the usage model. A second signal (an expiration signal) may be provided when the tracked cumulative usage time reaches a second predetermined level (X+n) based on the usage model.

Accordingly, the present disclosure provides a useful and ergonomic volatile substance dispenser. The dispenser accurately indicates to the consumer when it is time to replace the capsule. Moreover, the configuration of the indicator system is relatively simple, thereby providing efficiencies in computing power, manufacturing advantages, and more.

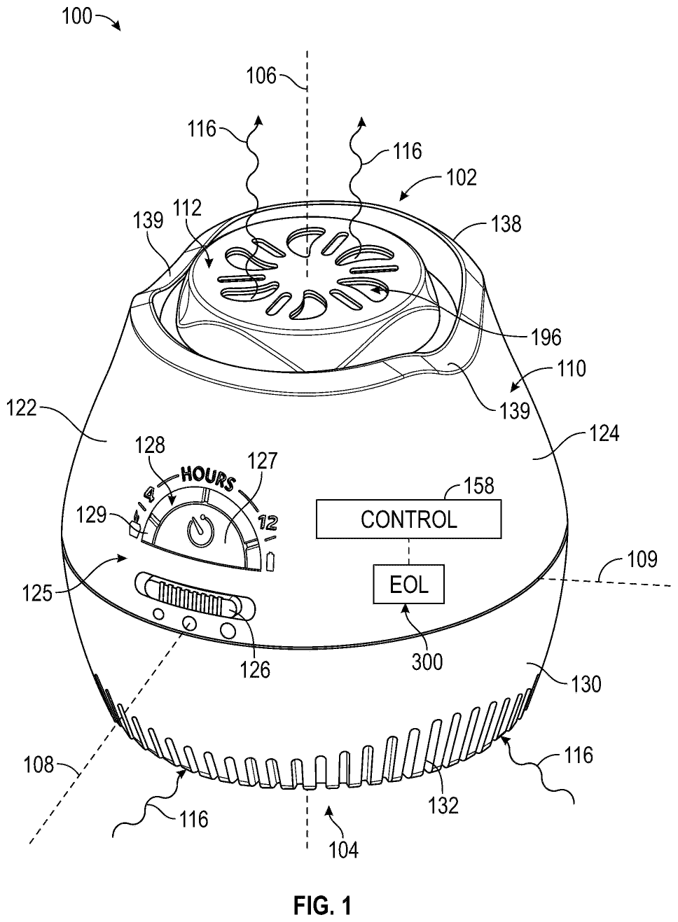

A volatile substance distribution system 100 will now be discussed according to example embodiments illustrated in FIG. 1. Generally, the system 100 includes an upper end 102 and a lower end 104 and a longitudinal axis 106 that extends therebetween. It will be appreciated that the terms “upper” and “lower” are relative terms based on the orientation shown in the Figures and are merely used as an example. Accordingly, the upper end 102 may be referred to as a “first end” and the lower end 104 may be referred to as a “second end.” A first radial axis 108 and a second radial axis 109 are also indicated in relation to the longitudinal axis 106 for reference purposes.

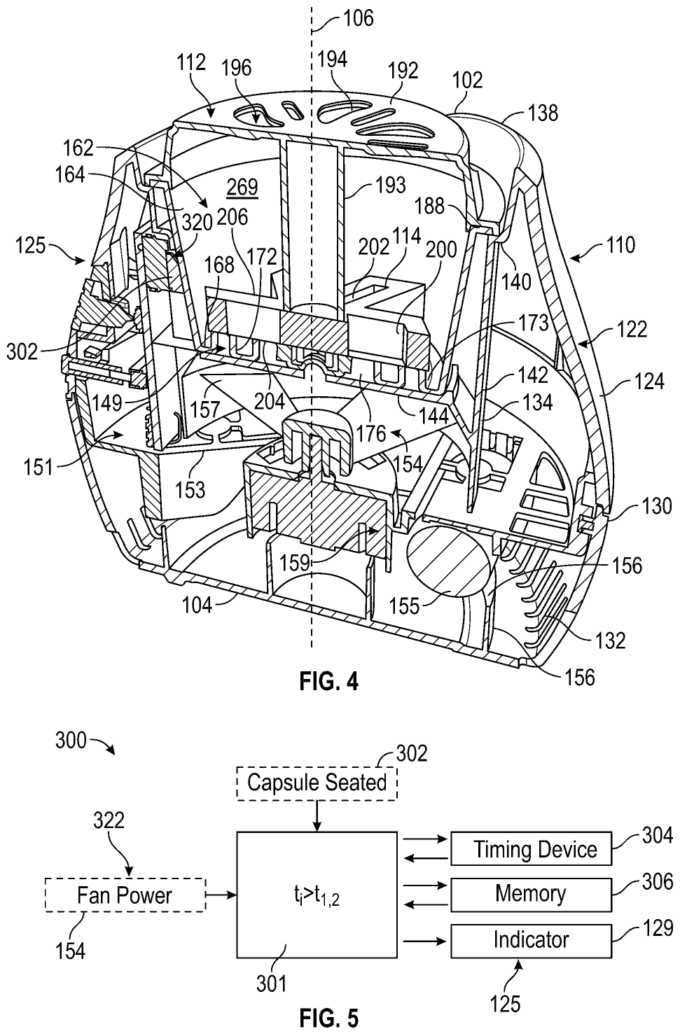

The volatile substance distribution system 100 may include a base unit 110 (FIGS. 1, 2, and 4) and at least one volatile substance capsule 112 (FIGS. 1, 2, and 4). In the illustrated embodiments, the base unit 110 may be configured for supporting a single capsule 112; however, in other embodiments, the base unit 110 may be configured for supporting multiple capsules 112. In some embodiments, the capsule 112 is a replaceable unit that may be removably supported by the base unit 110. The capsule 112 may also be referred to as a refill unit, as a cup or other container, as a pod, or as another term. The capsule 112 may be a single-use, disposable container, or the capsule 112 may be a refillable/reusable container. The capsule 112 may also be recyclable in some embodiments. The system 100 may additionally include a volatile substance member 114 that is contained within the capsule 112 (FIG. 4). The volatile substance member 114 may include or contain or otherwise comprise a volatile substance, such as an air freshener, essential oil, perfume, aromatherapy or homeopathy substances, materials for malodor control, insect control substances, etc. The term “volatile substance” as used herein will be understood broadly to include substances that readily vaporize and/or move into the air. In some embodiments, the system 100 may be configured for volatile substances that vaporize and move into an airstream flowing through the capsule 112 at normal ambient temperatures. As represented in FIGS. 1 and 4 and as will be described in detail, the system 100 may operate with the base unit 110 driving airflow (represented by arrow 116) through the capsule 112. The airflow 116, therefore, may carry the volatile substance from the member 114 and distribute it throughout the air outside the capsule 112. However, it will be appreciated that the system 100 may be otherwise configured, for example, to include a heating system for heating and vaporizing volatile substances, a wick, and/or other elements for delivering volatile substances into the air.

Referring now to FIGS. 1, 2, and 4, the base unit 110 will be discussed in detail according to example embodiments. The base unit 110 may include a housing 122. The housing 122 may be a relatively thin-walled or shell-like rigid structure constructed from one or more pieces. The piece(s) of the housing 122 may define an outer side member 124, a bottom member 130, and an inner member 134.

The outer side member 124 may be frusto-conic in shape. The outer side member 124 may be substantially centered about the longitudinal axis 106. The outer side member 124 may taper outward in width as the outer side member 124 extends from the upper end 102 toward the lower end 104. The outer side member 124 may have an arcuate or rounded (e.g., circular, ovate, etc.) cross section taken perpendicular to the axis 106. The outer side member 124 may support a user interface 125, which may include one or more user input devices and/or one or more user output devices.

The bottom member 130 of the housing 122 may be rounded and bowl-shaped. The bottom member 130 may be fixedly attached to the lower rim of the outer side member 124 of the housing 122 and may define the lower end 104. The bottom member 130 may include a relatively flat or otherwise supportive bottom surface for standing the bottom base unit 110 upright. The bottom member 130 may have a rounded cross section taken perpendicular to the longitudinal axis 106. In some embodiments, the width of the bottom member 130 (measured perpendicular to the axis 106) and the shape of the bottom member 130 may be configured for certain uses and environments. For example, the bottom member 130 may be sized and shaped to fit within a standard vehicle cupholder. Thus, the rounded shape and relatively small width may allow the base unit 110 to be securely received in the cup holder and the system 100 can freshen air within a vehicle.

The bottom member 130 may also include a plurality of apertures 132 (first apertures or inlet apertures). The apertures 132 may be elongate slots that extend through the thickness of the bottom member 130. In some embodiments, the apertures 132 may provide an inlet passage for the airflow 116 into the base unit 110.

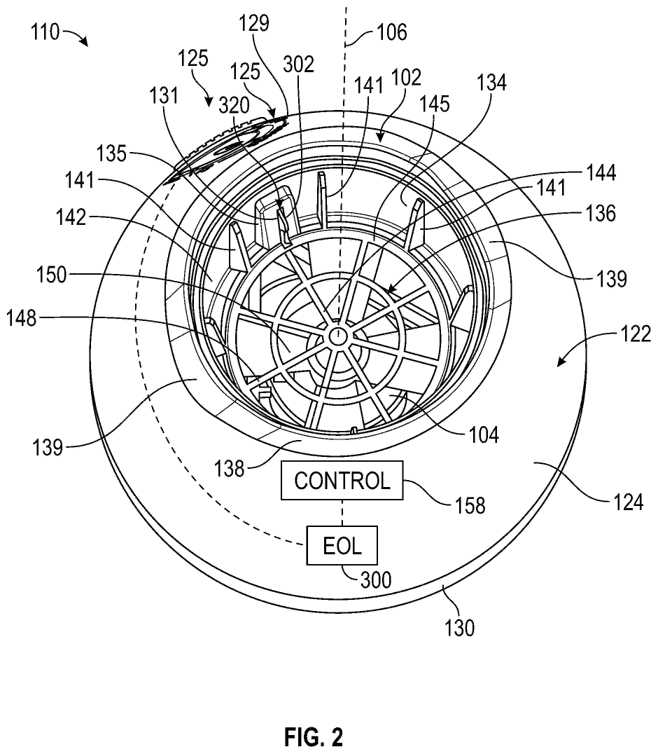

As shown in FIG. 2, the inner member 134 of the housing 122 may be cup-shaped and may be attached to the outer side member 124 along an upper rim 138 of the end 102. The inner member 134 may define a receptacle 136 of the housing 122. The receptacle 136 may be open at the upper end 102. The receptacle 136 may extend from the upper rim 138 and may be recessed therefrom, toward the lower end 104 along the axis 106. The receptacle 136 may be centered about the axis 106. The receptacle 136 may be shaped and sized according to the capsule 112. Thus, in some embodiments, the receptacle 136 may define a rounded cup-like recess for receiving the slightly-smaller capsule 112. The depth of the receptacle 136 may be sufficient to receive the majority of the capsule 112. For example, as shown in FIGS. 1 and 4, the receptacle 136 may be deep enough such that capsule 112 is nested with the upper rim and topside of the capsule 112 remaining exposed. The receptacle 136 may also be referred to as a docking station for the capsule 112.

In some embodiments, the upper rim 138 may include at least one notch 139. As shown in FIGS. 1 and 2, there may be two notches 139 that are spaced apart on opposite sides of the axis 106. The upper rim 138 may be scalloped with gradual contours to define the notches 139. The notches 139 may provide access to the capsule 112 for grasping and removing the capsule 112 from the base unit 110.

The inner member 134 of the housing 122 may include an inner ledge 140 (FIG. 4) that is disposed downward axially from the upper rim 138. The inner ledge 140 may extend substantially perpendicular to the axis 106 and inward radially toward the axis 106. The inner ledge 140 may be annular and may extend about the axis 106. The inner member 134 and the receptacle 136 may include a side wall 142, which may be substantially cylindrical, and which may depend downward along the axis 106 from the ledge 140. As shown in FIG. 2, the inner member 134 may include a plurality of elongate ribs 141 that extend longitudinally along the side wall 142 and that project slightly inward radially toward the axis 106. The ribs 141 may be spaced apart substantially equally in the circumferential direction about the axis 106. Additionally, the inner member 134 and the receptacle 136 may include a lower support 144 (FIG. 2). The lower support 144 may be a frusto-conic platform that is attached to the side wall 142 and that extends inwardly therefrom. The lower support 144 may include an outer ledge 145 and a plurality of elongate support members 148 that extend radially inward from the outer ledge 145 and that are connected at a central hub, for example, in a web-shaped arrangement.

The base unit 110 may further include an air outlet 150 that is defined between the elongate support members 148. The air outlet 150 may be in fluid communication with the interior of the housing 122 and with the apertures 132 of the bottom member 130. As such, the airflow 116 may move through the base unit 110 from the apertures 132 (the inlet), through the housing 122, and out of the housing 122 via the air outlet 150. As will be discussed, the air outlet 150 may blow air out of the base unit 110, upward along the axis 106, and into the capsule 112 in a downstream flow direction through the capsule 112.

The inner member 134 of the housing 122 may additionally include an opening, such as a slot 131 (FIG. 2). The slot 131 may be elongate and directed longitudinally, and the slot 131 may extend through the thickness of the inner member 134. The slot 131 may be axially straight. The slot 131 may extend from the outer ledge 145 and partially upward along the side wall 142. In some embodiments, the inner member 134 may include an inset portion 135 that is substantially rectangular, that surrounds the slot 131, and that is inset radially inward from surrounding portions of the side wall 142.

The base unit 110 may further include an abutment member 302 (FIG. 2) that is moveably disposed within the slot 131. The abutment member 302 may be supported for movement between a neutral position (FIG. 2) and an actuated position (FIG. 4). When positioned, the capsule 112 may abut against the abutment member 302 for detecting that the capsule 112 is seated and/or engaged with the base unit 110 and is ready for use as will be discussed.

The base unit 110 may additionally include an internal chassis 151 (FIG. 4). The chassis 151 may be flat and plate-like and may include a plurality of openings, surface features, etc. that will be discussed. The chassis 151 may be constructed of a rigid material, such as a polymeric material, and the chassis 151 may be constructed of the same material as the other members of the housing 122 in some embodiments. The chassis 151 may be disposed substantially perpendicular to the axis 106 and may extend laterally across the interior of the housing 122. The chassis 151 may be attached at its periphery to the outer side member 124 and/or to the bottom member 130.

The chassis 151 may include an underside 153 that supports one or more batteries 155. The underside 153 may include retaining members, electrical terminals, and/or other features for arranging the batteries 155 in a compact manner. For example, in some embodiments, there may be three batteries 155, which are arranged end-to-end in a triangular formation centered about the axis 106. This arrangement may evenly distribute weight of the batteries 155 to provide stability to the system 100 and prevent tipping.

As shown in FIG. 4, the interior side of the bottom member 130 may include projecting structures 156, such as walls, fins, posts, or other structures that project upwardly. The structures 156 may be annular in some embodiments. The structures 156 may support the batteries 155 and hold the batteries 155 to the chassis 151 in some embodiments. The underside 153 may also include a central cavity 159.

The base unit 110 may further include a fan 154. The fan 154 may be an electrical fan with a motor supported within the central cavity 159. The fan 154 may be compact and may have relatively low power requirements so that it can be battery-powered. The fan 154 may include a rotor 157 that extends through the chassis 151. The rotor 157 may be supported for rotation about the axis 106, and the blades of the rotor 157 drive the airflow 116 through the housing 122 and toward the capsule 112 via the air outlet 150. The chassis 151 may include a plurality of apertures through which the fan 154 may draw air. The rotor 157 may be supported for rotation about the axis 106 to draw the airflow 116 radially into the base unit 110 via the apertures 132 in the bottom member 130, through the chassis 151, and out via the air outlet 150, generally along the axis 106.

It will be appreciated that the system 100 may configured differently for moving air through the capsule 112. For example, instead of or in addition to the fan 154 the system 100 may incorporate an air pump, moveable bellows, air multipliers, or other features. Additionally, the fan 154 may be positioned differently from the illustrated embodiments without departing from the scope of the present disclosure. Moreover, as represented by the illustrated embodiment, the fan 154 may be configured for positive displacement relative to the capsule 112 such that the fan 154 drives (blows) the airflow 116 into the capsule 112. However, it will be appreciated that the fan 154 of the system 100 may be configured for negative displacement relative to the capsule 112 such that the fan 154 drives (sucks) air through the capsule 112. Moreover, instead of or in addition to the fan 154, the system 100 may include other features for moving volatiles out of the capsule 112, such as a heating element, etc. Furthermore, the system 100 may be configured for delivering volatiles passively and without relying on a power source to input power.

As mentioned above, the base unit 110 may include a user interface 125. The user interface 125 may have a variety of configurations without departing from the scope of the present disclosure. For example, as shown in FIG. 1, the user interface 125 may include one or more input devices 126, 127 and at least one output device 128.

In some embodiments, a first input device 127 may be a button. In some embodiments, the first input device 127 may be pressed once to turn ON the fan 154 and keep the fan 154 rotating continuously for a predetermined time interval (e.g., continuously for four hours) before being automatically shut OFF. Additionally, the first input device 127 may be pressed a second time to turn ON the fan 154 and keep the fan 154 rotating continuously for a second predetermined time interval (e.g., continuously for twelve hours). Furthermore, the first input device 127 may be pressed a third time to manually turn OFF the fan 154.

Furthermore, in some embodiments, a second input device 126 may be sliding switch that may be actuated for changing dispersion intensity of the volatile materials from the system 100. In some embodiments, the second input device 126 may be actuated for changing the speed of the fan between various speed settings, thereby changing dispersion intensity by the system 100.

Also, the output device 128 may include at least one visual output device 129 (FIG. 1). The visual output device 129 may include one or more lamps, LEDs, etc. There may be a plurality of different output devices 129 for indicating different information about the system 100. Also, in some embodiments a single output device 129 may provide a plurality of different signals that indicate different information about the system 100. It will be appreciated that the output device 128 may include an audio output device or other output device without departing from the scope of the present disclosure. Accordingly, the output device 128 may indicate that the fan 154 is ON. The output device 128 may also be configured for indicating whether power levels are low (e.g., to indicate that batteries should be changed). Furthermore, as will be discussed, the output device 128 may be configured for indicating when to change the capsule 112.

The base unit 110 may house a control system 158 within the housing 122. The control system 158 may be of a variety of types and may have a wide range of capabilities without departing from the scope of the present disclosure. In some embodiments, the control system 158 may include a processor, a memory device, sensor(s), and/or other components of a known computerized control system. Furthermore, the control system 158 may rely on programmed logic, sensor input, and/or stored data for controlling one or more features of the system 100.

For example, the control system 158 may be operably connected to the fan 154 for turning the fan 154 ON and OFF. In some embodiments, the control system 158 may be operably attached to the input device 127 to turn the fan 154 ON and OFF according to the user's input. In some embodiments, the user may input a first command (e.g., a first push of the input device 127), and the control system 158 may, in turn, continuously run the fan 154 for a first time interval (e.g., for four hours) before automatically shutting OFF the fan 154. Additionally, the user may input a second command (e.g., a second push of the input device 127), and the control system 158 may, in turn, continuously run the fan 154 for a second time interval (e.g., for twelve hours) before automatically shutting OFF the fan 154. The user may input a third command (e.g., a third push of the input device 127) to manually shut OFF the fan 154. The control system 158 may also adjust the speed of the fan 154 between two or more predetermined speed settings (e.g., Low speed, Medium speed, and High speed) based on the position of the second input device 126.

Referring now to FIGS. 1, 3, and 4, the capsule 112 will be discussed in detail according to example embodiments. The capsule 112 may include a housing 162, which houses the volatile substance member 114 (FIG. 4). The housing 162 may be hollow and cup-shaped. In some embodiments, the housing 162 may be substantially cylindrical and may have a generally circular cross section taken normal to the axis 106. The housing 162 may be centered on the axis 106 and may extend along the axis 106 between a first end 161 (i.e., a bottom or inlet end) and a second end 163 (i.e., a top or outlet end). The first end 161 may be oriented toward the lower end 104 and the second end 163 may be disposed proximate the upper end 102 when mounted on the base unit 110.

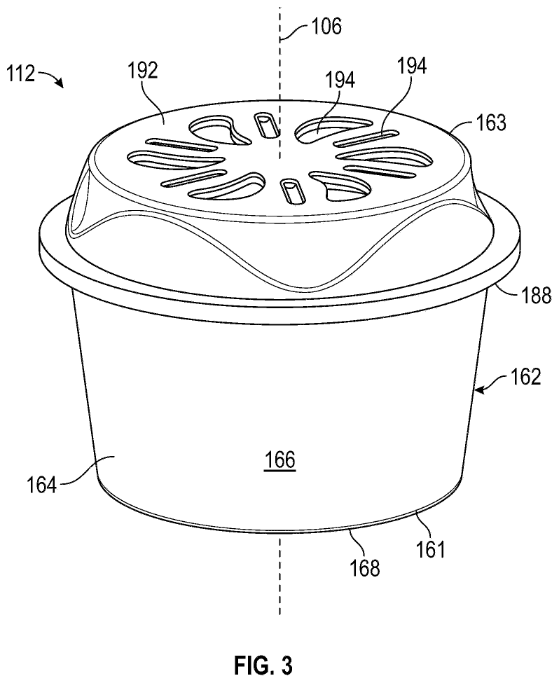

As shown in FIG. 3, the housing 162 may generally include a cup member 164 and a cover member 192. The cup member 164 and cover member 192 may cooperate to house, encapsulate, and/or retain the volatile substance member 114 therein.

The cup member 164 may be a unitary member made of a polymeric material. The cup member 164 may be somewhat flexible but may be rigid enough to support itself and contents therein. The cup member 164 may include an outer wall 166 that extends circumferentially about the longitudinal axis 106. The outer wall 166 may be centered on the axis 106. The outer wall 166 may also extend along the longitudinal axis 106 in a first direction (downward) toward the first end 161 and may terminate at a first terminal end 168 of the capsule 112. The outer wall 166 may also include an upper rim 188, which is spaced apart longitudinally from the first terminal end 168 of the capsule 112. The outer wall 166 may have a circular cross section taken normal to the axis 106. In other embodiments, the outer wall 166 may have a different shape, such as a square or other polygonal shape. The outer wall 166 may be frusto-conic and tapered slightly with respect to the axis 106. As such, the outer wall 166 proximate the first end 161 may be narrower than the outer wall 166 proximate the second end 163.

The cup member 164 may include a lower platform 172, which is disposed proximate the first terminal end 168. The lower platform 172 may span across the first terminal end 168 and may be attached at its periphery to the outer wall 166. The lower platform 172 may be offset in the longitudinal direction from the first terminal end 168 so as to define an annular trough 173 at the periphery of the lower platform 172 and proximate the outer wall 166. The lower platform 172 may define an air inlet 176 (e.g., at least one opening) extending therethrough in the axial direction. The lower platform 172 may support the volatile substance member 114 thereon such that air passing through the air inlet 176 flows over and past the volatile substance member 114.

The cover member 192 may be a frusto-conic disc that is attached at its periphery to the upper rim 188 of the cup member 164. The cover member 192 may be made of a polymeric material. In some embodiments, the cover member 192 may be welded (i.e., plastic welded) to the cup member 164, although it will be appreciated that the cover member 192 may be adhesively attached or otherwise fastened to the cup member 164 without departing from the scope of the present disclosure. The cover member 192 may include a plurality of apertures 194. The apertures 194 may have a variety of shapes without departing from the scope of the present disclosure, such as slot-shaped apertures 194, teardrop shaped apertures 194, or other shapes. As will be discussed, the apertures 194 may define an outlet port 196 for the capsule 112.

A part of the volatile substance member 114 is shown in FIG. 4 according to example embodiments. The volatile substance member 114 in some embodiments may include a substrate 200 with a volatile substance absorbed thereon. For example, the substrate 200 may be made from a sheet of material (e.g., cotton, paper, plant-based material, non-woven material, porous or spiralized plastic, polymeric material, corrugated sheet, sponge material, etc.) with fragrance oil thereon. Accordingly, the volatile substance member 114 may be substantially dry and moisture-free inside the capsule 112 during normal consumer use to avoid any spillage or leakage of fragrance oil. In other embodiments, the volatile substance member 114 and/or the substrate 200 may comprise beads, particles, etc. that are scented with a fragrance oil. In further embodiments, the volatile substance member 114 may include a container for a fragrant gel, fragrance oil, a wick, or other features without departing from the scope of the present disclosure.

The substrate 200 may be formed in a variety of shapes without departing from the scope of the present disclosure. For example, the substrate 200 may be arranged substantially in a star-shape that is centered on the axis 106. The substrate 200 may include a first side 204 and a second side 206. The first side 204 may face the lower end 104, and the second side 206 may face the upper end 102. A plurality of through-ways 202 may be defined through the volatile substance member 114 along the axis 106 from the first side 204 to the second side 206. The volatile substance member 114 may also be heart-shaped, rectangular, triangular, or shaped otherwise.

The volatile substance member 114 may be supported atop the lower platform 172 of the cup member 164 and may be centered thereon. Also, the cover member 192 may include a projecting member 193 (FIG. 4) that projects and depends from the inside top surface to abut against the second side 206 of the volatile substance member 114. Accordingly, the lower platform 172 and the projecting member 193 may cooperate to retain the volatile substance member 114 in place.

The first side 204 and the second side 206 may be open such that air passing through the capsule 112 may pass over and through the volatile substance member 114. Accordingly, there may be a relatively high amount of exposed surface area for passing the volatile substance to the airflow 116.

To use the system 100, packaging may be removed from the capsule 112. For example, packaging, covering, seals, etc. may be removed from the capsule 112. In some embodiments, the capsule 112 may include at least one peel-off seal that covers over the openings in the first end 161 and the second end 163.

Then, the capsule 112 may be placed on and may be engaged with the base unit 110 (i.e., moved to an engaged position with the base unit 110 as shown, for example, in FIGS. 1 and 4). Specifically, the capsule 112 may be centered with respect to the axis 106 and dropped into the receptacle 136. As shown in FIG. 4, the upper rim 188 of the capsule 112 may rest on the inner ledge 140 when seated in the receptacle 136. Also, the taper dimension of the ribs 141 may substantially correspond to the taper of the outer wall 166 of the capsule 112 such that the outer wall 166 lies against and snugly nests against the ribs 141 on the side member 124 of the base unit 110. Also, the size and shape of the circular terminal end 168 of the capsule 112 may correspond to that of the outer ledge 145 of the base unit 110 such that the terminal end 168 snugly fits and nests on the outer ledge 145 of the base unit 110. Accordingly, the capsule 112 and the receptacle 136 may correspond in shape and size. Both the receptacle 136 and the housing 162 of the capsule 112 may be cup-shaped with rounded (e.g., circular) cross sections taken normal to the axis 106. Both the receptacle 136 and the capsule 112 may be aligned and centered on the axis 106 with corresponding widths (i.e., diameters) and tapered surfaces. As such, the capsule 112 may nest within the receptacle 136 and may be secured therein.

Furthermore, as shown in FIG. 4, an airflow fluid coupling 149 may be established between the capsule 112 and the base unit 110 as a result of the capsule 112 engaging with the base unit 110. Specifically, the air outlet 150 of the base unit 110 may fluidly connect to the air inlet 176 of the capsule 112 when the capsule 112 is supported within the receptacle 136. Placement of the capsule 112 on the base unit 110 may coincidentally fluidly connect and align the air inlet 176 to the air outlet 150 of the base unit 110. In some embodiments, the air inlet 176 covers over an entirety of the air outlet 150 of the base unit 110. Stated differently, the air inlet 176 surrounds of the base unit 110 with respect to the axis 106 (e.g., the air inlet 176 encircles the air outlet 150). Also, the terminal end 168 seats against the outer ledge 145 to block leakage flow between the outside of the capsule 112 and the base unit 110. In this position, the receptacle 136, the air outlet 150, the first end 161 of the capsule 112, the air inlet 176, the second end 163, and the outlet port 196 may be coaxial and centered with respect to the longitudinal axis 106. Also, in this position, the air outlet 150, and the air inlet 176 may be substantially aligned along the longitudinal axis 106.

Then, the fan 154 may be turned ON by the control system 158. For example, the user may push the input device 127, and the control system 158 may command the rotor 157 to begin rotating the rotor 157 of the fan 154 for a set time period. The fan 154 may draw air into the inlet apertures 132 and blow the air out of air outlet 150. The airflow 116 may be received and directed by the air inlet 176 and into the housing 162 of the capsule 112. The airflow 116 may be directed into the through-ways 202 of the volatile substance member 114. The airflow 116 may, therefore, pass through the member 114, into a so-called headspace 269 of the capsule 112 defined axially between the volatile substance member 114 and the cover member 192 of the capsule 112. The airflow 116 may eventually exit the capsule 112 via the apertures 194. As long as the rotor 157 of the fan 154 is powered ON, the airflow 116 may be continuously driven from the inlet apertures 132 of the base unit 110 and out of the capsule 112 via the apertures 194, and volatile material from the member 114 may be carried away into the surrounding air.

After the predetermined time period, the control system 158 may automatically turn the fan 154 OFF. If needed, the user may use the input device 127 to “manually” turn the fan 154 OFF, for example, by pressing the input device 127 multiple times (e.g., three time) in quick succession. The capsule 112 may remain in the receptacle 136 and engaged with the base unit 110 while the fan 154 is OFF. As such, the capsule 112 can remain in standby for when the fan 154 is again turn ON for delivering the volatiles.

As represented in FIGS. 1, 2, and 5, the system 100 may further include an indicator system 300 configured to indicate a usage condition of the capsule 112. The indicator system 300 may indicate that the capsule 112 is at and/or near the end of its useful life and, as such, may be referred to as an end-of-life indicator system 300. Accordingly, the indicator system 300 may assist the user and indicate when the capsule 112 should be replaced. Also, as will be explained, the indicator system 300 includes a number of features that provide ergonomic advantages, improved usefulness, manufacturing efficiencies, and more.

As represented in FIGS. 2, 4, and 5, the indicator system 300 may include a capsule sensor 320 that is configured to detect the presence of the capsule 112. The capsule sensor 320 may detect that the capsule 112 is in the removably engaged position, and that the capsule 112 remains seated in the receptacle 136.

In some embodiments, the capsule sensor 320 may comprise the abutment member 302 (FIGS. 2 and 4) as discussed above. The abutment member 302 may be a lever that is rotationally mounted within the housing 122. The abutment member 302 may be biased (e.g., by a mechanical spring) toward its neutral position (FIG. 2) in which the abutment member 302 projects inwardly radially from the inset portion 135 of the side wall 142. When the capsule 112 is placed in the receptacle 136, the outer wall 166 of the capsule 112 may abut against the member 302 and push it radially outward, allowing the outer wall 166 to rest against the inset portion 135. The abutment member 302 may be operably attached to electronics (e.g., a switch that switches between OPEN and CLOSED based on the position of the abutment member 302). Thus, as represented in FIG. 5, the capsule sensor 320 may detect that the capsule 112 is seated and may provide a corresponding signal to a processor 301 of the control system 158. If the capsule 112 should be removed from the base unit 110, then the abutment member 302 may bias outward and the capsule sensor 320 no longer provides the signal.

Although a mechanical switch (i.e., the abutment member 302) is described above, it will be appreciated that the capsule sensor 320 may be differently configured without departing from the scope of the present disclosure. For example, the presence of the capsule 112 may be detected by an electronic sensor with a light beam that is broken by the presence of the capsule 112 on the base unit 110. The base unit 110 may also include an electronic reader that scans a barcode, an RFID chip, or other identifier on the capsule 112.

The indicator system 300 may also include a fan sensor 322. The fan sensor 322 may be an electronic sensor that detects usage of the fan 154. The fan sensor 322 may, in some embodiments, determine whether power is being sent to the fan 154 to rotate the rotor 157. In other embodiments, the fan sensor 322 may be a motion sensor that detects whether the rotor 157 is rotating. The fan sensor 322 may be configured in other ways as well without departing from the scope of the present disclosure. The fan sensor 322 may provide at least one signal to the processor 301 for distinguishing whether the fan 154 is in an operating condition (e.g., power to rotor 157) or a nonoperating condition (e.g., no power to rotor 157).

The indicator system 300 may further include a timing device 304 as represented in FIG. 5. The timing device 304 may operate as a timer or stopwatch. The timing device 304 may be operatively connected to the processor 301 for tracking and measuring the amount of time that the fan 154 is operating while the capsule remains in the engaged position on the base unit 110.

Furthermore, the indicator system 300 may include a computerized memory device 306. The computerized memory device 306 may store various data. For example, the computerized memory device 306 may store the actual, tracked usage time measured by the timing device 304. The memory device 306 may also store one or more predetermined usage thresholds (e.g., threshold usage times) that the processor 301 may compare with actual usage times that are detected by the timing device 304.

The indicator system 300 may additionally include an output device, such as the visual output device 129 (e.g., a lamp) of the user interface 125 described above. The output device 129 may be operatively attached to the processor 301, which may command the output device 129 to provide one or more signals indicative of a usage condition of the capsule 112. In other embodiments of the indicator system 300, the output device may include a speaker for outputting an audio indication about the capsule usage. In some embodiments, the output device 129 may provide a first signal indicating that the capsule 112 is nearly used up and almost expended, and the output device 129 may provide a second signal indicating that the capsule 112 has been used up. Specifically, in some embodiments, the output device 129 may be a lamp that remains unlit when the capsule 112 is new, the same lamp may flash when the capsule is nearly used up, and the same lamp may remain lit when the capsule has been used up.

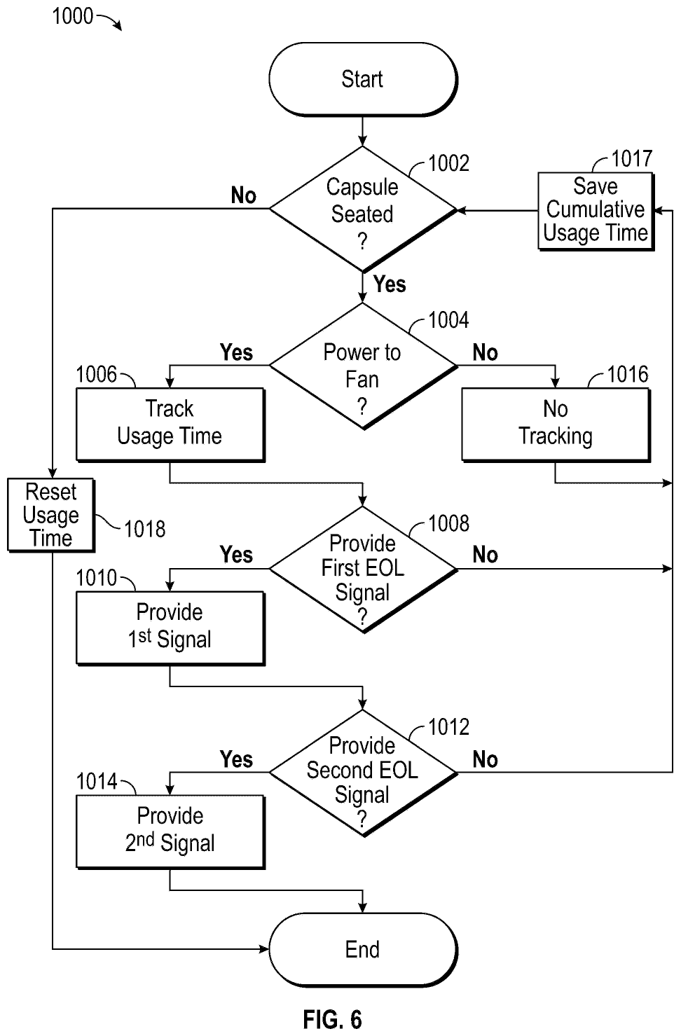

Referring now to FIG. 6, a method 1000 of operating the system 100 is shown according to example embodiments. The method 1000 may begin at 1002, wherein it is determined whether the capsule 112 is seated in the base unit 110 (i.e., whether the capsule 112 is in the engaged position). As discussed above, the method 1000 at 1002 may be affirmatively answered if the capsule 112 is pressing against the abutment member 302 and maintaining it in the actuated position (FIG. 4). The capsule sensor 320 may provide the corresponding signal to the processor 301 as shown in FIG. 5.

If 1002 is answered affirmatively, the method 1000 may continue at 1004, wherein it is determined whether the fan 154 is operational and the rotor 157 is being driven in rotation. As shown, at 1004 of the method 1000, the fan sensor 322 may determine whether or not power is being supplied to the rotor 157 of the fan 154, and the fan sensor 322 may provide a corresponding signal to the processor 301.

If 1004 is answered affirmatively, the method 1000 may continue at 1006, wherein the processor 301 may generate and send a control command to the timing device 304 to begin to track the amount of time that the fan 154 is operating (i.e., the amount of time that the rotor 157 is being driven in rotation).

Next, the method 1000 may continue at 1008, wherein the processor 301 determines whether a first end-of-life signal is to be provided by the output device 129. For example, the processor 301 may access the memory device 306, which stores a first time threshold, t₁. The first time threshold, t₁, may correspond to an amount of usage at which the capsule 112 is nearly expired. Thus, at 1008, the processor 301 may compare the first time threshold, t₁, to the cumulative amount of usage time, t₁, being tracked by the timing device 304 beginning at 1006. If the cumulative amount of usage time, t₁, exceeds the first threshold (i.e., tᵢ>t₁), then 1008 may be answered affirmatively, and the method 1000 may continue at 1010. At 1010 of the method, the processor 301 may generate and send a control signal to send the first indication (i.e., that the capsule 112 is almost expired). In some embodiments, this first indication may be the output device 129 flashing on and off intermittently.

Subsequently, the method 1000 may continue at 1012, wherein the processor 301 determines whether a second end-of-life signal is to be provided by the output device 129. For example, the processor 301 may access the memory device 306, which stores a second time threshold, t₂. The second time threshold, t₂, may correspond to an amount of usage at which the capsule 112 is estimated to be expired. Thus, at 1008, the processor 301 may compare the second time threshold, t₂, to the cumulative amount of usage time, t₁, being tracked by the timing device 304 beginning at 1006. If the cumulative amount of usage time, t₁, exceeds the second threshold (i.e., t₁>t₂), then 1012 may be answered affirmatively, and the method 1000 may continue at 1014. At 1014 of the method, the processor 301 may generate and send a control signal to send the second indication (i.e., that the capsule 112 is expired). In some embodiments, the second indication may be the output device 129 remaining continuously lit. Then, the method 1000 may terminate.

As shown in FIG. 6, if 1004 is answered negatively (e.g., the fan sensor 322 detects power is no longer sent to the fan 154, detects the rotor 157 is no longer driven, etc.), then at 1016, the timing device 304 may stop timing and tracking usage. In other words, the timing device 304 may toll while the fan 154 is OFF and the capsule 112 remains engaged. Then, at 1017, the cumulative (total) amount of usage time may be recorded in the memory device 306. The method 1000 may loop back to 1002 as shown in FIG. 6. If the capsule 112 remains seated (i.e., 1002 answered affirmatively), and power is again supplied to the fan 154 (i.e., 1004 answered affirmatively), then the timing device 304 may restart the tracking and, at 1017, the cumulative amount of usage time stored in memory may be updated.

If 1008 is answered negatively, then the method 1000 may loop back to 1002. Similarly, if 1012 is answered negatively, then the method 1000 may loop back to 1002.

If, at some point, 1002 is answered negatively (e.g., the capsule 112 is removed and disengaged from the base unit 110), then the method 1000 may continue at 1018. At 1018, the processor 301 may reset the cumulative amount of usage time that is stored in the memory device 306. Then, the method 1000 may terminate.

It will be appreciated that the indicator system 300 may indirectly detect the remaining amount of volatile material in the capsule 112 and determine whether to provide the indicators accordingly. The indicator system 300 may be programmed according to an approximation of the rate of dispensing of the volatile material over time. For example, the capsule 112 may be assumed to deliver volatile material and to expire after twenty-four hours of continuous operation of the fan 154. Thus, the second threshold, t₂, in some embodiments may be twenty-four hours of fan use (t₂=24 hours). The first threshold, t₁, may be more arbitrary. For example, the first threshold, t₁, may be set at eighteen hour (t₁=18 hours). In some embodiments, the indicator system 300 ignores the fan speed or intensity setting (adjusted using the second input device 126). This allows computing power to remain low while also providing accurate indications as to usage of the capsule 112. However, it will be appreciated that the indicator system 300 may account for the fan speed/intensity setting and/or other control settings in some embodiments of the present disclosure.

Terms such as “first” and “second” have been utilized above to describe similar features or characteristics (e.g., longitudinal directions) in view of the order of introduction during the course of description. In other sections of this Application, such terms can be varied, as appropriate, to reflect a different order of introduction. While at least one exemplary embodiment has been presented in the foregoing Detailed Description, it should be appreciated that a vast number of variations exist. It should also be appreciated that the exemplary embodiment or exemplary embodiments are only examples, and are not intended to limit the scope, applicability, or configuration of the present disclosure in any way. Rather, the foregoing Detailed Description will provide those skilled in the art with a convenient road map for implementing an exemplary embodiment of the present disclosure. It is understood that various changes may be made in the function and arrangement of elements described in an exemplary embodiment without departing from the scope of the present disclosure as set forth in the appended claims.

What is claimed is:

1. A volatile substance distribution system comprising:
a housing configured to removably receive a capsule such that the capsule is in an engaged position with the housing, the capsule containing a volatile substance member;
a fan supported by the housing and selectively variable between a nonoperating condition and an operating condition, the fan, in the operating condition and with the capsule in the engaged position, driving air through the housing and the capsule;
an indicator system configured to indicate a usage condition of the capsule, the indicator system including a capsule sensor, a timing device, and an output device;
the capsule sensor configured to detect that the capsule is in the engaged position;
the timing device configured to track a cumulative usage time of the capsule according to an amount of time the fan is in the operating condition with the capsule in the engaged position as detected by the capsule sensor; and
the output device configured to provide an indication of the usage condition when the cumulative usage time reaches a predetermined threshold time.

2. The volatile substance distribution system of claim 1, wherein the output device is configured to provide a first indication when the cumulative usage time reaches a first predetermined threshold time; and
wherein the output device is configured to provide a second indication when the cumulative usage time reaches a second predetermined threshold time.

3. The volatile substance distribution system of claim 1, further comprising a control system that controls the fan so that, in the operating condition, a rotor of the fan drivingly rotates for a preset time interval before an automatic shutoff of the rotor to the nonoperating condition.

4. The volatile substance distribution system of claim 3, wherein the control system has a first user setting and a second user setting;
wherein the control system controls the fan so that, in the first user setting, the rotor drivingly rotates for a first time interval before the automatically shutoff to the nonoperating condition; and
wherein the control system controls the fan so that, in the second user setting, the rotor drivingly rotates for a second time interval before the automatically shutoff to the nonoperating condition.

5. The volatile substance distribution system of claim 3, further comprising a user control for manual shutoff of the rotor to the nonoperating condition.

6. The volatile substance distribution system of claim 1, further comprising a memory device that stores the tracked cumulative usage time of the capsule.

7. The volatile substance distribution system of claim 6, further comprising a control system that resets the tracked cumulative usage time stored in the memory device is reset as a result of the capsule sensor detecting that the capsule is removed from the engaged position.

8. The volatile substance distribution system of claim 1, wherein housing includes a receptacle that removably receives the capsule in the engaged position with the housing, the receptacle being aligned with the fan such that the capsule is aligned with the fan when in the engaged position; and
wherein the capsule sensor includes an abutment member disposed within the receptacle and configured to abut against the capsule when the capsule is in the engaged position.

9. The volatile substance distribution system of claim 8, wherein the housing supports the abutment member for movement between a neutral position and an actuated position, the abutment member configured to abut against the capsule and move from the neutral position toward the actuated position as the capsule is moved toward the engaged position, and wherein the abutment member is configured to remain in abutting contact with the capsule in the actuated position as the capsule remains in the engaged position.

10. The volatile substance distribution system of claim 9, wherein the fan and the receptacle are centered with respect to an axis, wherein the receptacle includes a recess defined by a side wall and a lower support, the axis extending through the lower support, and wherein the abutment member projects inwardly from the side wall with respect to the axis when in the neutral position.

11. The volatile substance distribution system of claim 9, wherein the abutment member is biased toward the neutral position.

12. The volatile substance distribution system of claim 1, wherein the output device is a visual output device that provides a visual indication of the usage condition when the cumulative usage time reaches the predetermined threshold time.

13. The volatile substance distribution system of claim 12, wherein the visual output device provides a first visual indication when the cumulative usage time reaches a first predetermined threshold time; and
wherein the visual output device provides a second visual indication when the cumulative usage time reaches a second predetermined threshold time.

14. A method of operating a volatile substance distribution system comprising:
engaging a capsule and a base unit in an engaged position, the capsule containing a volatile substance member and being removably engageable with the base unit, and the base unit including a housing that supports a fan, the fan selectively variable between a nonoperating condition and an operating condition, the fan, in the operating condition and with the capsule in the engaged position, driving air through the housing and the capsule; and
indicating, with an indicator system of the base unit, a usage condition of the capsule, including:
detecting, with a capsule sensor, that the capsule is in the engaged position;
tracking, with a timing device, a cumulative usage time of the capsule according to an amount of time the fan is in the operating condition with the capsule in the engaged position as detected by the capsule sensor; and
indicating, with an output device, the usage condition when the cumulative usage time reaches a predetermined threshold time.

15. The method of claim 14, wherein indicating the usage condition includes:
providing, with the output device, a first indication when the cumulative usage time reaches a first predetermined threshold time; and
providing, with the output device, a second indication when the cumulative usage time reaches a second predetermined threshold time.

16. The method of claim 14, further comprising drivingly rotating a rotor of the fan in the operating condition for a preset time interval before the automatic shutoff of the rotor to the nonoperating condition.

17. The method of claim 16, further comprising drivingly rotating the rotor for a first time interval before the automatic shutoff of the rotor the nonoperating condition; and
further comprising drivingly rotating the rotor for a second time interval before the automatic shutoff of the rotor to the nonoperating condition.

18. The method of claim 14, further comprising storing the tracked cumulative usage time of the capsule in a memory device.

19. The method of claim 18, further comprising resetting the tracked cumulative usage time stored in the memory device as a result of the capsule sensor detecting that the capsule is removed from the engaged position.

20. The method of claim 14, further comprising, when the fan is in the operating condition, receiving a manual shutoff user input and, in response, shutting off the fan from the operating condition to the nonoperating condition.
